(19) United States
(12) Patent Application Publication
(10) Pub. No.: US 2025/0074912 A1
(43) Pub. Date: Mar. 6, 2025

(54) IMPROVED PROCESS FOR THE PREPARATION OF UPADACITINIB

(71) Applicant: MSN LABORATORIES PRIVATE LIMITED, R&D CENTER, Hayderabad, Telangana (IN)

(72) Inventors: Thirumalai Rajan SRINIVASAN, Hyderabad, Telangana (IN); Eswaraiah SAJJA, Hyderabad, Telangana (IN); Vijayavitthal T. MATHAD, Hyderabad, Telangana (IN); Rajeshwar Reddy SAGYAM, Hyderabad, Telangana (IN); Venkata Narasayya SALADI, Hyderabad (IN); Navin Kumar Reddy KESHAVAREDDY, Hyderabad, Telangana (IN); Balraju KAMMARI, Hyderabad, Telangana (IN); Raghavendar Reddy PARIGI, Hyderabad, Telangana (IN); Venkata Chary UTNOORI, Hyderabad, Telangana (IN); Murali YATA, Hyderabad, Telangana (IN)

(73) Assignee: MSN LABORATORIES PRIVATE LIMITED, R&D CENTER, Hyderabad, Telangana (IN)

(21) Appl. No.: 18/727,092

(22) PCT Filed: Jan. 6, 2023
(86) PCT No.: PCT/IN2023/050016

§ 371 Date: Jul. 7, 2024

(30) Foreign Application Priority Data
Jan. 6, 2022 (IN) 202241000792
Mar. 18, 2022 (IN) 202241015009
Nov. 9, 2022 (IN) 202241064007

(51) Int. Cl. C07D 471/14; A61K 31/437; C07C 67/52; C07C 69/78; C07C 201/16; C07C 205/57

(52) U.S. Cl. CPC C07D 471/14; A61K 31/437; C07C 67/52; C07C 69/78; C07C 201/16; C07C 205/57; C07B 2200/13; C07C 2601/16

(57) ABSTRACT

The present invention relates to an improved process for the preparation of (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide formula-1.

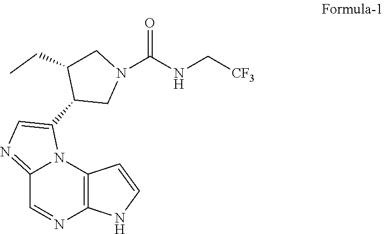 [SMILES: CC[C@@H]1CN(C(=O)NCC(F)(F)F)C[C@@H]1c1cnc2cnc3[nH]ccc3n12]

The present invention relates to crystalline form of 8-((3R,4S)-4-ethylpyrrolidin-3-yl)-3H-imidazo[1,2-a]pyrrolo[2,3-e]pyrazine compound of formula-2.

The present invention also relates dibenzoyl-L-tartaric acid salt and 4-nitrobenzoic acid salt of (3S,4R)-3-ethyl-4-(3H-imidazo[1,2-a]pyrrolo[2,3-e]pyrazin-8-yl)-N-(2,2,2-trifluoro ethyl)pyrrolidine-1-carboxamide and its polymorph forms which are useful in the preparation of pure Upadacitinib. The present invention also relates to a crystalline form of Upadacitinib tartrate and its process thereof.

### RELATED APPLICATION

This application claims the benefit of priority of our Indian patents application Ser. Nos. 20/224,1000792 filed on 6 Jan. 2022 and 202241015009 filed on 18 Mar. 2022 and 202241064007 filed 9 Nov. 2022, which is incorporated herein by reference.

### FIELD OF THE INVENTION

The present invention relates to an improved process for the preparation of (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide formula-1

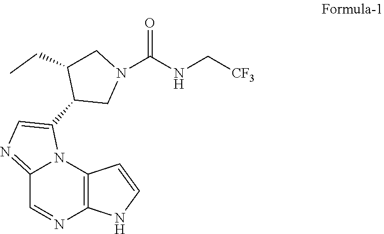 [SMILES: CC[C@@H]1CN(C(=O)NCC(F)(F)F)C[C@@H]1c1cnc2cnc3[nH]ccc3n12]

### BACKGROUND OF THE INVENTION

Upadacitinib is chemically known as (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide.

Upadacitinib is a Janus kinase (JAK) inhibitor & indicated for the treatment of adults with moderately to severely active rheumatoid arthritis who have had an inadequate response or intolerance to methotrexate, and it is approved by USFDA under brand name of RINVOQ.

U.S. Pat. No. 8,426,411 B2 (hereinafter referred as US'411) discloses a process for preparation of Upadacitinib as shown in below.

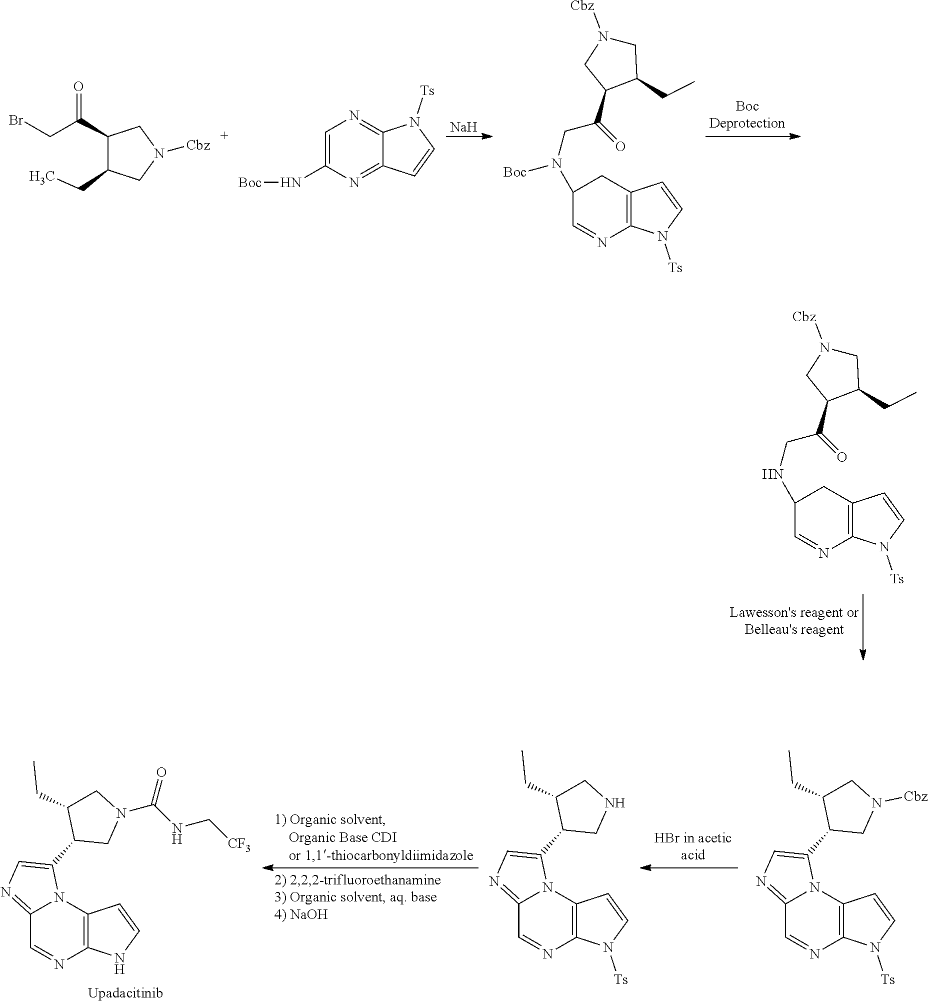

PCT publication, WO2017/066775 A1 discloses processes for the preparation of Upadacitinib as shown in below.

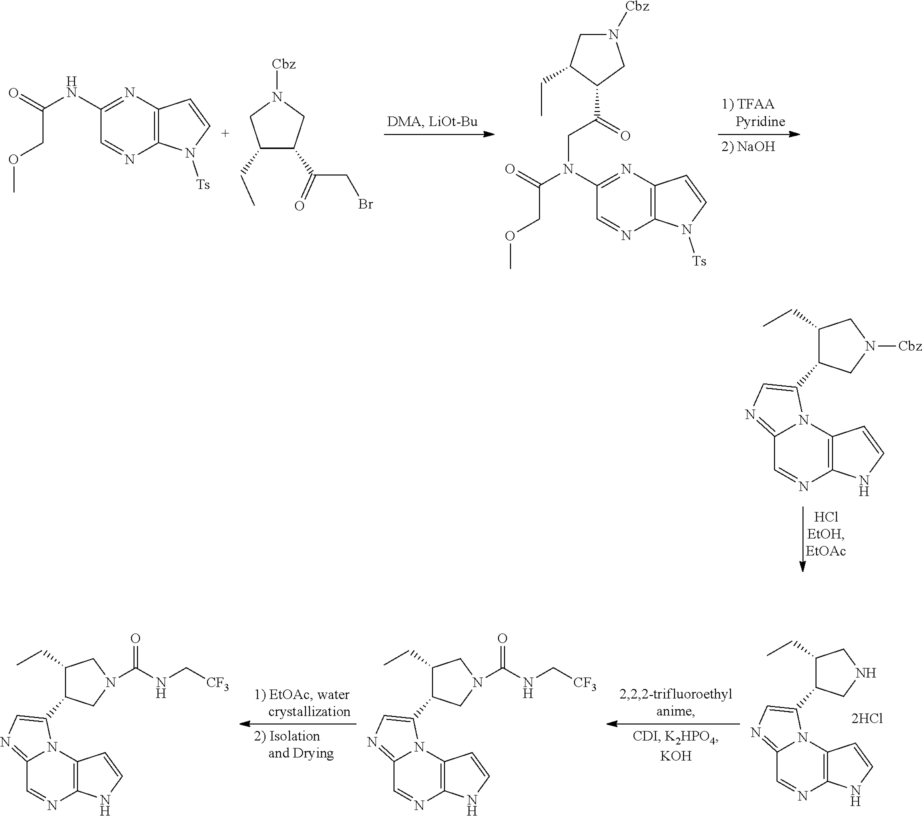

The above processes suffers from several drawbacks that includes use of sodium hydride, Lawesson's reagent and corrosive acids, which are not suggestable and making the process not viable on commercial scale.

CN10369659 B discloses process for the preparation of 8-((3R,4S)-4-ethylpyrrolidin-3-yl)-3H-imidazo[1,2-a]pyrrolo[2,3-e]pyrazine comprising dissolving (3S,4R)-benzyl 3-ethyl-4-(3-tosyl-3H-imidazo[1,2-a]pyrrolo[2,3-e]pyrazin-8-yl)pyrrolidine-1-carboxylate in tetrahydrofuran and then adding sodium hydroxide and followed by reducing the obtained compound using Pd/C to provide 8-((3R,4S)-4-ethylpyrrolidin-3-yl)-3H-imidazo[1,2-a]pyrrolo[2,3-e]pyrazine with 55% yield.

Therefore, in order to overcome the imperfections of the prior art processes, inventors of the present invention has developed an improved process for the preparation of Upadacitinib, which is eco-friendly, uses cheaper and mild reagents and provides Upadacitinib with good yield and purity.

### Advantages of the Present Invention

Avoids use of palladium and sodium hydride reagents.
Avoids pressure reactions.
Avoids cumbersome workups and column purifications.
Involves mild reagents and cheaper reagents and making the process simple and easy to scale up.
Cost-effective, improved yield, least number of stages and increases productivity.

### BRIEF DESCRIPTION OF THE INVENTION

The present invention relates to an improved process for the preparation of Upadacitinib.

The present invention relates to crystalline form of 8-((3R,4S)-4-ethylpyrrolidin-3-yl)-3H-imidazo[1,2-a]pyrrolo[2,3-e]pyrazine.

The present invention also relates to novel salts of Upadacitinib, polymorphic forms and processes for the preparation thereof.

The present invention relates to a crystalline form of Upadacitinib tartrate herein after designated as Form-M.

The present invention relates to process for the preparation of a crystalline form-M of Upadacitinib tartrate.

### BRIEF DESCRIPTION OF THE DRAWINGS

FIG. 1: Illustrates the PXRD pattern of crystalline form of 8-((3R,4S)-4-ethylpyrrolidin-3-yl)-3H-imidazo[1,2-a]pyrrolo[2,3-e]pyrazine.

FIG. 2: Illustrates the PXRD pattern of crystalline Form-I of dibenzoyl-L-tartaric acid of Upadacitinib.

FIG. 3: Illustrates the PXRD pattern of crystalline form of 4-nitrobenzoic acid salt of Upadacitinib.

FIG. 4: Illustrates the PXRD pattern of crystalline Form-II of dibenzoyl-L-tartaric acid of Upadacitinib.

FIG. 5: Illustrates the PXRD pattern of crystalline Form-M of Upadacitinib tartrate.

FIG. 6: Illustrates the PXRD pattern of amorphous Upadacitinib.

### DETAILED DESCRIPTION OF THE INVENTION

The term solvent used in the present invention refers to “hydrocarbon solvents” such as n-hexane, n-heptane, cyclohexane, petroleum ether, benzene, toluene, xylene and the like; “ether solvents” selected from dimethyl ether, diisopropyl ether, diethyl ether, methyl tert-butyl ether, 1,2-dimethoxy ethane, tetrahydrofuran, trifluoroacetic anhydride, 1,4-dioxane and the like; “ester solvents” selected from methyl acetate, ethyl acetate, isopropyl acetate, n-butyl acetate, isobutyl acetate and the like; “polar-aprotic: solvents selected from dimethylacetamide, dimethyl formamide, dimethylsulfoxide, N-methylpyrrolidone and the like; “chloro solvents” selected from dichloromethane, dichloroethane, chloroform, carbon tetrachloride and the like; “ketone solvents” selected from acetone, methyl ethyl ketone, methyl isobutyl ketone and the like; “nitrile solvents” selected from acetonitrile, propionitrile, isobutyronitrile and the like; “alcohol solvents” selected from methanol, ethanol, n-propanol, isopropanol, n-butanol, isobutanol, t-butanol, n-pentanol, isopentanol, 2-nitroethanol, ethylene glycol, 2-methoxyethanol, 1, 2-ethoxyethanol, diethylene glycol, 1, 2, or 3-pentanol, neo-pentyl alcohol, t-pentyl alcohol, diethylene glycol monoethyl ether, benzyl alcohol, phenol, or glycerol and the like; “polar-aprotic solvents” selected from dimethylacetamide (DMA), dimethylformamide (DMF), dimethylsulfoxide (DMSO), N-methylpyrrolidone (NMP) and the like; “polar solvents” selected from water or mixtures thereof.

As used herein the present invention the term base is selected from “alkali metal carbonates” selected from sodium carbonate, potassium carbonate, lithium carbonate and the like; “alkali metal bicarbonates” selected from sodium bicarbonate, potassium bicarbonate and the like; “alkali metal hydroxides” selected from sodium hydroxide, potassium hydroxide, lithium hydroxide barium hydroxide, cesium hydroxide, strontium hydroxide, calcium hydroxide, and the like; “alkali metal alkoxides” such as selected from sodium methoxide, sodium ethoxide, potassium methoxide, potassium ethoxide, sodium tert.butoxide, potassium tert.butoxide and the like; “alkali metal hydrides” selected from sodium hydride, potassium hydride and the like; “alkali metal amides” selected from sodium amide, potassium amide, lithium amide, lithium diisopropyl amide (LDA) and the like; “alkali metal phosphates” selected from disodium hydrogen phosphate, dipotassium hydrogen phosphate and “organic bases” like methyl amine, diisopropyl amine, diisopropylethyl amine, diisobutylamine, triethylamine, tert.butyl amine, pyridine, 4-dimethylaminopyridine (DMAP), N-methylmorpholine (NMM), 1,8-diazabicyclo[5.4.0]undec-7-ene (DBU), 1,5-diazabicyclo[4.3.0]non-5-ene (DBN), 1,4-diaza bicyclo[2.2.2]octane (DABCO), imidazole or mixtures thereof.

A solid-state form (or polymorph) are referred to herein as polymorphically pure or as substantially free of any other solid state (or polymorphic) forms. As used in this context, the expression “substantially free of any other forms” will be understood to mean that the solid state form contains about 20% or less, about 10%> or less, about 5% or less, about 2% or less, about 1%) or less, about 0.5% or less, about 0.1%> or less or 0% of any other forms of the subject compound as measured, for example, by XRPD. Thus, solid state of Upadacitinib described herein as substantially free of any other solid state forms would be understood to contain greater than about 80% (w/w), greater than about 90% (w/w), greater than about 95% (w/w), greater than about 98%) (w/w), greater than about 99% (w/w), greater than about 99.5% (w/w), greater than about 99.9%) (w/w) of the subject solid-state form of Upadacitinib.

The high-performance liquid chromatography (referred to hereinafter as HPLC) has usually been utilized as the technique to analyze a trace amount of ingredient contained in a sample.

A “pure compound” as used herein is meant to cover compounds with a purity of at least about 95%, or more preferred at least about 97%, or more preferred at least about 99%, or more preferred at least about 99.6% and even more preferred at least about 99.9% as measured by HPLC.

Wherein, P₁, P₂ or Pg are refers to protecting groups and wherein, P₁ and P₂ protecting groups are selected from same or different protecting groups.

In first embodiment, the present invention provides an improved process for the preparation of Upadacitinib, comprising:
a) treating the compound of general formula-3 with a suitable base in a suitable solvent to provide compound of formula-2,

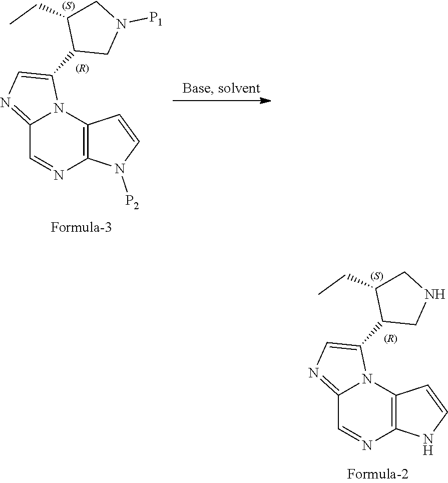

b) converting the compound of formula-2 to provide Upadacitinib.
wherein, P₁ and P₂ refers to a protecting group selected from carbobenzyloxy (Cbz), benzoyl (Bz), benzyl (Bn), tosyl (Ts), p-methoxybenzyl carbonyl, tert-butyloxycarbonyl (BOC), acetyl (Ac), carbamate, p-methoxybenzyl, 3,4-dimethoxybenzyl, p-methoxyphenyl (PMP), trichloroethylchloroformate, nosyl and the like thereof.

In the process of the first embodiment, wherein the suitable solvent used in step-a) is selected from ketone solvents, nitrile solvents, alcohol solvents, ester solvents, ether solvents, polar solvents and the like, preferably, isopropanol and water.

In the process of the first embodiment, wherein the suitable base is used in step-a) is selected from inorganic bases such as sodium hydroxide, potassium hydroxide, cesium carbonate, sodium carbonate, potassium carbonate, lithium carbonate, rubidium carbonate, sodium hydroxide, potassium hydroxide, lithium hydroxide, barium hydroxide, barium hydroxide octa hydrate, cesium hydroxide, strontium hydroxide, calcium hydroxide, Pyridine and the like, preferably, barium hydroxide octa hydrate.

In first aspect of first embodiment, the present invention provides an improved process for the preparation of Upadacitinib, comprising:
a) reacting (3S,4R)-benzyl 3-ethyl-4-(3-tosyl-3H-imidazo[1,2-a]pyrrolo[2,3-e]pyrazin-8-yl)pyrrolidine-1-carboxylate of formula-3a with barium hydroxide in isopropanol and water to provide 8-((3R,4S)-4-ethylpyrrolidin-3-yl)-3H-imidazo[1,2-a]pyrrolo[2,3-e] pyrazine of formula-2,

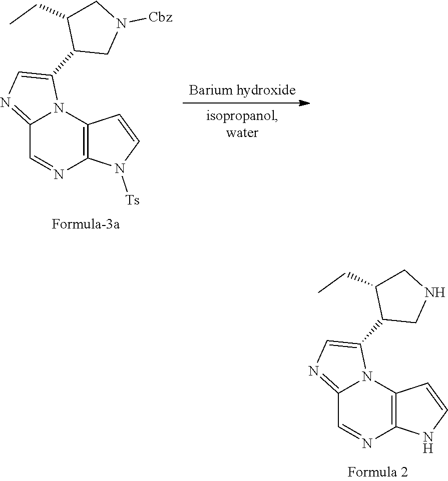

b) converting the compound of formula-2 to provide Upadacitinib.

Prior art process for the preparation of compound of formula-2 involves the usage of palladium for the conversion of formula-3 to compound of formula-2, which is expensive and commercially not viable. Whereas, the present invention involves usage of mild and cheaper barium hydroxide for the removal of protecting groups.

In second embodiment, the present invention provides an improved process for the preparation of Upadacitinib, comprising:
a) reacting the compound of general formula-3 (i) with a suitable base in a suitable solvent to provide compound of formula-2,

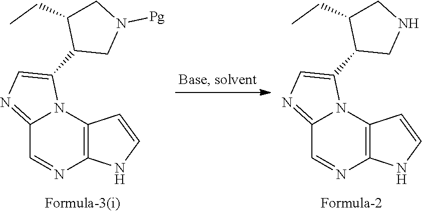

b) converting the compound of formula-2 to provide Upadacitinib.
   wherein, Pg refers to a protecting group selected from carbobenzyloxy (Cbz), benzoyl (Bz), benzyl (Bn), tosyl (Ts), p-methoxybenzyl carbonyl, tert-butyloxycarbonyl (BOC), acetyl (Ac), carbamate, p-methoxybenzyl, 3,4-dimethoxybenzyl, p-methoxyphenyl (PMP), trichloro ethyl chloroformate, nosyl and the like thereof.

In the process of the second embodiment, wherein the suitable solvent used in step-a) is selected from ketone solvents, nitrile solvents, alcohol solvents, ester solvents, ether solvents, polar solvents and the like, preferably, isopropanol and water.

In the process of the second embodiment, wherein the suitable base is used in step-a) is selected from inorganic bases such as sodium hydroxide, potassium hydroxide, cesium carbonate, sodium carbonate, potassium carbonate, lithium carbonate, rubidium carbonate, sodium hydroxide, potassium hydroxide, lithium hydroxide, barium hydroxide, barium hydroxide octa hydrate, cesium hydroxide, strontium hydroxide, calcium hydroxide, pyridine and the like, preferably, barium hydroxide octa hydrate.

In first aspect of second embodiment, the present invention provides an improved process for the preparation of Upadacitinib, comprising:
a) reacting (3S,4R)-benzyl 3-ethyl-4-(3H-imidazo[1,2-a]pyrrolo[2,3-e]pyrazin-8-yl) pyrrolidine-1-carboxylate of formula-3a (i) with barium hydroxide in isopropanol and water to provide 8-((3R,4S)-4-ethylpyrrolidin-3-yl)-3H-imidazo[1,2-a]pyrrolo[2,3-e] pyrazine of formula-2,

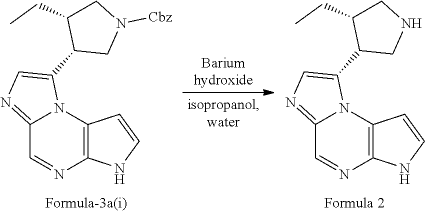

b) converting the compound of formula-2 to provide Upadacitinib.

The process according to the present invention wherein, barium hydroxide octa hydrate used may range from 2 moles to 20 moles ratio per 1 mole of compound of general formula-3.

The process according to the present invention wherein, barium hydroxide octa hydrate used preferably in the range from 8 mole to 16 mole ratio per 1 mole of compound of general formula-3.

In third embodiment, the present invention provides crystalline form of 8-((3R,4S)-4-ethylpyrrolidin-3-yl)-3H-imidazo[1,2-a]pyrrolo[2,3-e]pyrazine compound of formula-2.

In first aspect of the third embodiment, the present invention provides crystalline form of 8-((3R,4S)-4-ethylpyrrolidin-3-yl)-3H-imidazo[1,2-a]pyrrolo[2,3-e]pyrazine compound of formula-2 which is herein after referred as Form-M.

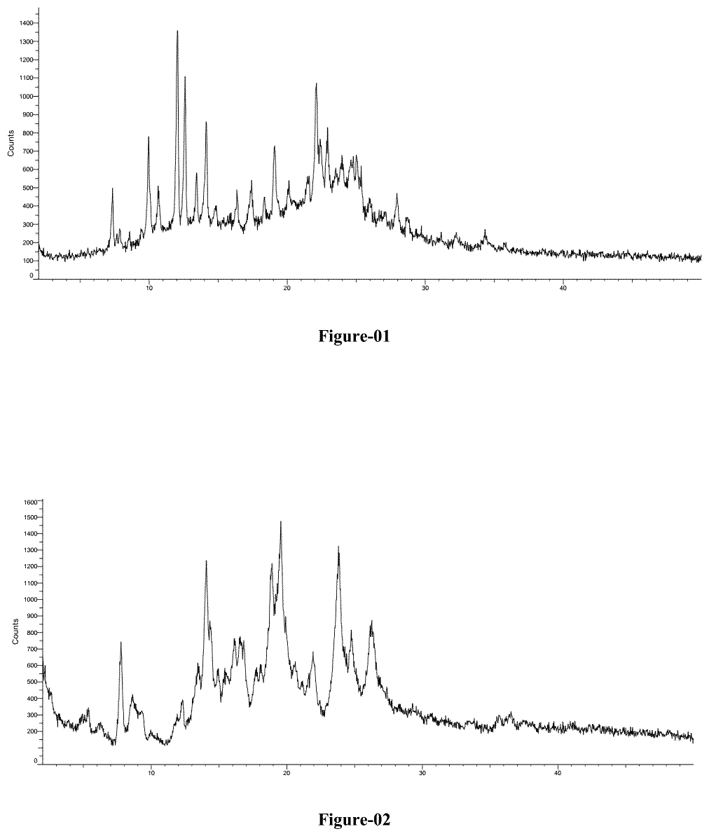

In second aspect of the third embodiment, the present invention provides crystalline form-M of 8-((3R,4S)-4-ethylpyrrolidin-3-yl)-3H-imidazo[1,2-a]pyrrolo[2,3-e]pyrazine compound of formula-2 is characterized by powder X-Ray diffractogram as illustrated in FIG. 1.

The process according to the present invention wherein, 8-((3R,4S)-4-ethylpyrrolidin-3-yl)-3H-imidazo[1,2-a]pyrrolo[2,3-e]pyrazine having purity of at least about 98.80% as measured by HPLC.

Compounds of formula-3 or formula-3a or formula-3a(i) used in the present invention can be prepared by the process described in U.S. Pat. No. 8,426,411 B2 or any of the prior known processes.

In an embodiment, 8-((3R,4S)-4-ethylpyrrolidin-3-yl)-3H-imidazo[1,2-a]pyrrolo[2,3-e] pyrazine of formula-2 obtained according to the present invention is having Imidazolo impurity, Imidazolo Isomer impurity, Acetyl impurity, Des tosyl impurity, Tosyl Des Cbz impurity, Des Cbz isomer impurity, Benzyl alcohol impurity and PTSA impurity less than 2% as measured by HPLC.

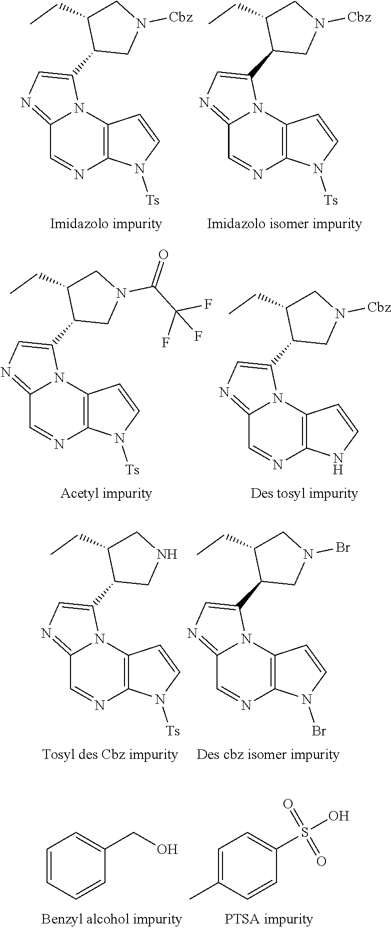

The process according to the present invention wherein, converting the compound of formula-2 to Upadacitinib is carrying out by the process as described in scheme or exemplified in the present invention.

In fourth embodiment, the present invention provides dibenzoyl-L-tartaric acid salt of Upadacitinib.

In fifth embodiment, the present invention provides dibenzoyl-L-tartaric acid salt of Upadacitinib.

In first aspect of the fifth embodiment, the present invention provides solid-state forms of dibenzoyl-L-tartaric acid salt of Upadacitinib.

The second aspect of the fifth embodiment, the present invention provides crystalline Form-I of dibenzoyl-L-tartaric acid salt of Upadacitinib is characterized by its powder X-Ray diffractogram as illustrated in FIG. 2.

The third aspect of the fifth embodiment, the present invention provides crystalline Form-II of dibenzoyl-L-tartaric acid salt of Upadacitinib is characterized by its powder X-Ray diffractogram as illustrated in FIG. 4.

In sixth embodiment, the present invention provide a process for the preparation of pure Upadacitinib, comprising treating the salts of Upadacitinib with a suitable base to provide pure Upadacitinib of formula-1.

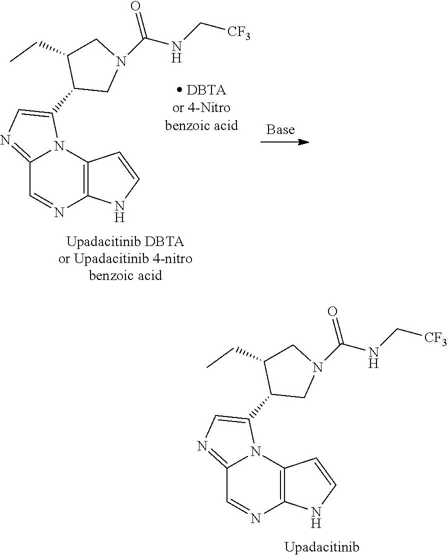 [SMILES: CC[C@@H]1CN(C(=O)NCC(F)(F)F)C[C@@H]1c1cnc2cnc3[nH]ccc3n12 ; CC[C@@H]1CN(C(=O)NCC(F)(F)F)C[C@@H]1c1cnc2cnc3[nH]ccc3n12]

In the process of the sixth embodiment, wherein the suitable solvent used is selected from ketone solvents, nitrile solvents, alcohol solvents, ester solvents, chloro solvents, hydrocarbon solvents, polar aprotic solvents, ether solvents and polar solvents like water or mixture thereof; the suitable base is selected from organic base or inorganic base.

The first aspect of the sixth embodiment, the present invention provides a process for the preparation of crystalline Form-I of dibenzoyl-L-tartaric acid salt of Upadacitinib, comprising:
a) treating the compound of formula-1 with dibenzoyl-L-tartaric acid in isopropanol, water and isopropyl acetate,
b) heating the mixture obtained in step-a) to a temperature ranging from 25° C. to 60° C.,
c) isolating crystalline Form-I of dibenzoyl-L-tartaric acid salt of Upadacitinib.

The second aspect of the sixth embodiment, the present invention provides a process for the preparation of crystalline Form-II of dibenzoyl-L-tartaric acid salt of Upadacitinib, comprising:
a) treating the compound of formula-1 with dibenzoyl-L-tartaric acid in acetonitrile and water,
b) heating the mixture obtained in step-a) to a temperature ranging from 25° C. to 65° C.,
c) isolating crystalline Form-II of dibenzoyl-L-tartaric acid salt of Upadacitinib.

In the process of sixth embodiment, isolating the crystalline forms of dibenzoyl-L-tartaric acid salt of Upadacitinib is carried out by employing any of the techniques known in the art or isolating the crystalline forms of dibenzoyl-L-tartaric acid salt of Upadacitinib by the decantation, filtration by gravity or suction, centrifugation, adding solvent to make slurry followed by filtration, or other techniques specific to the equipment used and the like, and optionally washing with a solvent.

In seventh embodiment, the present invention provides 4-nitrobenzoic acid salt of Upadacitinib.

In eighth embodiment, the present invention provides solid-state forms of 4-nitrobenzoic acid salt of Upadacitinib.

In first aspect of the seventh embodiment, the present invention provides crystalline form of 4-nitrobenzoic acid salt of Upadacitinib.

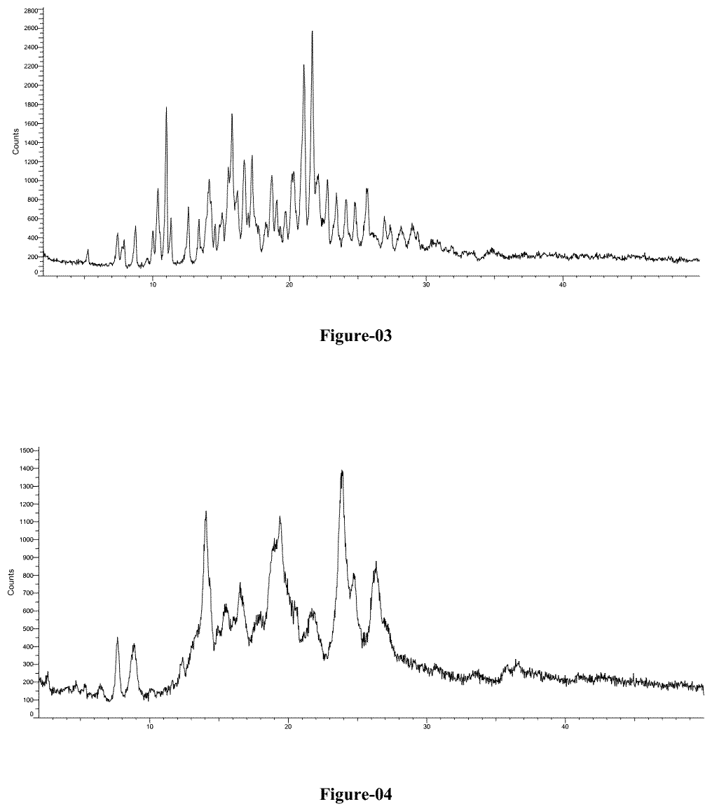

In second aspect of the seventh embodiment, the present invention provides crystalline form of 4-nitrobenzoic acid salt of Upadacitinib is characterized by powder X-Ray diffractogram as illustrated in FIG. 3.

In ninth embodiment, the present invention provides a process for the preparation of crystalline form of 4-nitrobenzoic acid salt of Upadacitinib, comprising:
a) treating the compound of formula-1 with 4-nitrobenzoic acid in acetonitrile to provide 4-nitrobenzoic acid salt of Upadacitinib,
b) isolating to provide crystalline form of 4-nitrobenzoic acid salt of Upadacitinib.

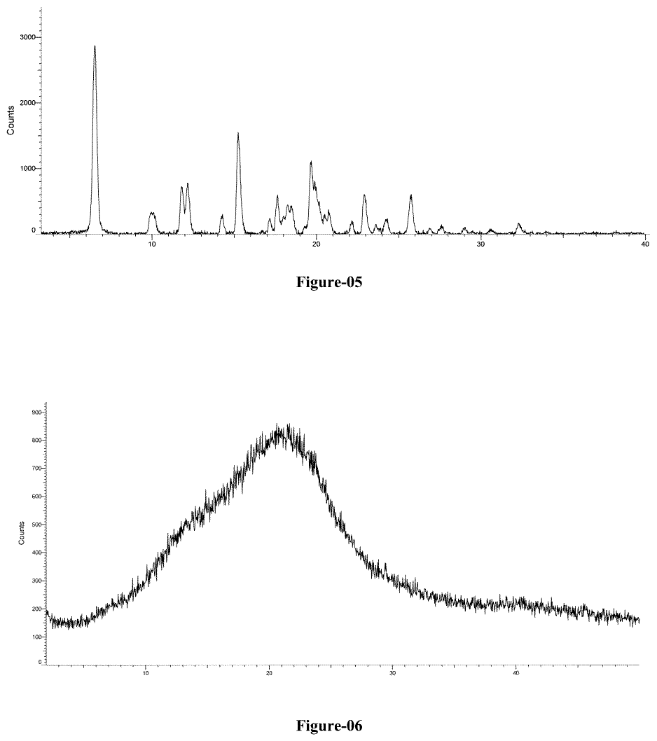

In tenth embodiment, the present invention provides the crystalline Form-M of Upadacitinib tartrate, is characterized by powder X-Ray diffractogram as illustrated in FIG. 5.

In eleventh embodiment, the present invention provides a process for the preparation of crystalline Form-M of Upadacitinib tartrate, which comprises:
a) providing a solution of Upadacitinib of formula-1,
b) providing a solution of tartaric acid,
c) adding the solution obtained in step-a) to the solution obtained in step-b) to provide crystalline Form-M of Upadacitinib tartrate.

In the process of eleventh embodiment, wherein the suitable solvent used in step-a) is ester solvent which is selected from methyl acetate, ethyl acetate, isopropyl acetate, n-butyl acetate, isobutyl acetate or mixture thereof.

In the process of eleventh embodiment, providing a solution of Upadacitinib of step-a) comprises dissolving Upadacitinib in ethyl acetate. Optionally filtering the mixture to make it particle free.

In the process of eleventh embodiment, wherein the suitable solvent used in step-b) is ether solvent is selected from dimethyl ether, diisopropyl ether, diethyl ether, methyl tert-butyl ether, 1,2-dimethoxyethane, tetrahydrofuran, ketone solvent selected from acetone, methyl ethyl ketone, methyl isobutyl ketone or mixture thereof.

In the process of eleventh embodiment in step-c), adding solution obtained in step-a) in solution obtained in step-b) at a temperature ranging from 0° C. to 15° C.

In the process of eleventh embodiment, isolating the crystalline Form-M of Upadacitinib tartrate can be carried out by any methods known in the art or isolating the crystalline form-M is employing any of the techniques, but not limited to: decantation, filtration by gravity or suction, centrifugation, adding solvent to make slurry followed by filtration, or other techniques specific to the equipment used and the like, and optionally washing with a solvent.

In the process of the present invention, drying the crystalline Form-M of Upadacitinib tartrate is carried out by suitable drying equipment such as tray dryer, vacuum oven, rotatory cone dryer, air oven, fluidized bed dryer, spin flash dryer, flash dryer, or the like. The drying is carried out at atmospheric pressure or under reduced pressure at temperature of less than about 100° C., less than about 60° C., less than about 40° C., or any other suitable temperature. The drying is carried out for any time period required for obtaining a desired quality, such as from about 15 minutes to 10 hours or longer.

The process of the present invention can be represented schematically as follows:

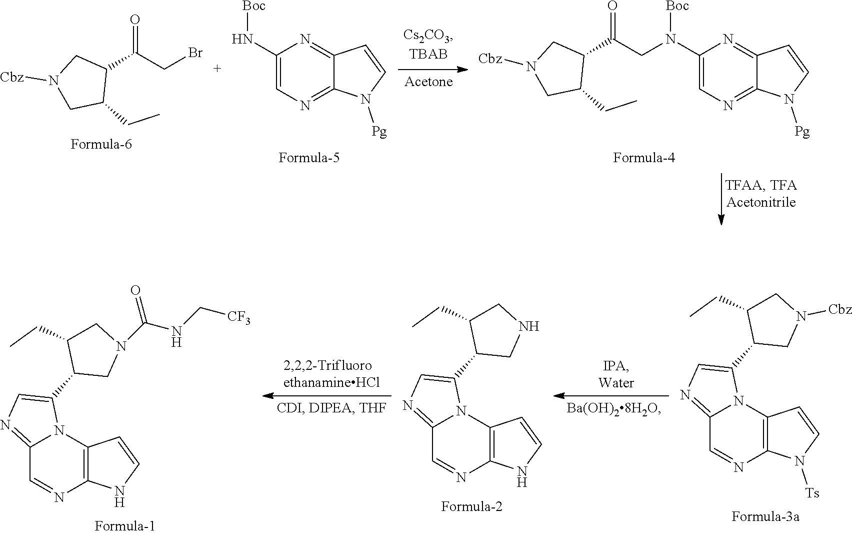

In twelfth embodiment, the present invention provides a process for the preparation of pure Upadacitinib of formula-1, comprises:
a) reacting compound of general formula-4 with trifluoro acetic acid and trifluoro acetic anhydride in acetonitrile in the absence of base to provide compound of general formula-3,
b) treating the compound of general formula-3 with a suitable base to provide compound of formula-2,
c) reacting the compound of formula-2 with 2,2,2-trifluoroethanamine hydrochloride in the presence of coupling agent and a base and followed by treating the obtained compound with an acid to provide acid addition salt of Upadacitinib,
d) treating the acid addition salt of Upadacitnib with a suitable base to provide pure Upadacitinib.

In the process of the twelfth embodiment, wherein the suitable base used in step-b) and step-d) is selected from sodium hydroxide, potassium hydroxide, cesium carbonate, sodium carbonate, potassium carbonate, lithium carbonate, rubidium carbonate, sodium hydroxide, potassium hydroxide, lithium hydroxide, barium hydroxide, barium hydroxide octa hydrate, cesium hydroxide, strontium hydroxide, calcium hydroxide, pyridine thereof;
the suitable coupling agent used in step-c) is selected from EDCI·HCl, HATU, DCC and CDI with or without HOBt or HOEt or in a combination thereof, the suitable base selected from triethyl amine, diisopropylethyl amine, dimethylaminopyridine;
the suitable acid used in step-c) is selected form dibenzoyl-L-tartaric acid and 4-nitrobenzoic acid;
the suitable solvent used in step-a) to step-d) is selected from ketone solvents, ester solvents, chloro solvents, alcohol solvents, ether solvents, nitrile solvents, polar aprotic solvents, hydrocarbon solvents and water or mixture thereof.

Upadacitinib obtained according to the present invention is having Des cbz impurity, 5E CDI adduct impurity, Upadacitinib diasteriomer impurity and Des tosyl impurity less than 0.15% as measured by HPLC.

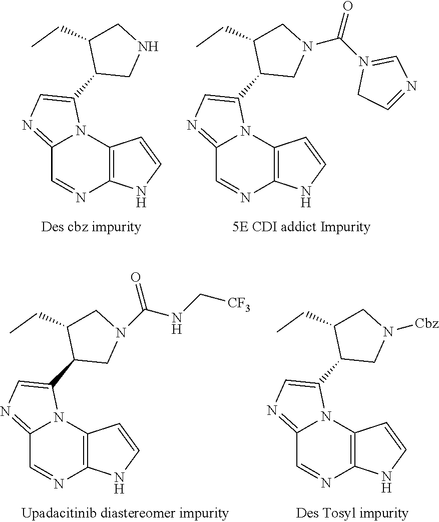

Upadacitinib obtained according to the present invention is having purity of at least about 95%; preferably of at least about 97%; more preferably of at least about 98%; most preferably of at least about 99.9% as measured by HPLC.

Upadacitinib obtained according to the present invention is having chiral purity of at least about 95%; preferably of at least about 97%; more preferably of at least about 98%; most preferably of at least about 99.9% as measured by HPLC.

Amorphous form of Upadacitinib obtained according to the present invention is having purity of greater than 99.80% as measured by HPLC.

Amorphous form of Upadacitinib obtained according to the present invention is stable for 12 months at 25° C./60+5% relative humidity and at 40±2° C./75±5% relative humidity.

Upadacitinib obtained according to the present invention is having particle size distribution D₉₀<250 μm.

Upadacitinib produced by the present invention is micronized or milled in a conventional technique to get the desired particle size to achieve desired solubility profile based on different forms of pharmaceutical composition requirements. Techniques that are used for particle size reduction include, but not limited to ball, roller and hammer mills, and jet mills. Milling or micronization is performed before drying, or after the completion of drying of the product.

In another embodiment, Upadacitinib obtained by the present invention can be used in the preparation of various pharmaceutical compositions formulated in a manner suitable for the route of administration to be used.

In yet another embodiment, the present invention encompasses pharmaceutical compositions comprising Upadacitinib tartrate obtained by the process of the present invention and one or more pharmaceutical acceptable excipient.

In yet another embodiment, the present invention also encompasses pharmaceutical compositions comprising Upadacitinib of formula-1 obtained by the process of the present invention and one or more pharmaceutical acceptable excipient. As used herein, the term “pharmaceutical compositions” or “pharmaceutical formulations” include tablets, pills, powders, liquids, suspensions, emulsions, granules, capsules, suppositories, or injection preparations.

### P-XRD Method of Analysis:

The powder X-ray diffraction (PXRD) analysis of compound obtained according to the present invention were carried out by using BRUKER/D8 ADVANCE or BRUKER/D2 PHASER diffractometer using CuKα radiation of wavelength 1.5406A°.

### PSD Method of Analysis:

Particle size distribution (PSD) analysis was performed using Malvern Mastersizer 2000 instrument.

The best mode of carrying out the present invention is illustrated by the below mentioned examples. These examples are provided as illustration only and hence should not be construed as limitation of the scope of the invention.

### EXAMPLES

### Example-1: Preparation of (3R,4S)-benzyl 3-(2-((tert-butoxycarbonyl)(5-tosyl-5H-pyrrolo[2,3-b]pyrazin-2-yl)amino)acetyl)-4-ethylpyrrolidine-1-carboxylate formula-4

Acetone (260 ml) was added to tert-butyl (5-tosyl-5H-pyrrolo[2,3-b] pyrazin-2-yl) carbamate (20 gms) of formula-5 at 25-30° C. and stirred for 10 min. Cesium carbonate (50.32 gms) and tetrabutylammonium bromide (8.3 gms) were added to the mixture at 25-30° C. and stirred for 1 hour. Molecular sieves were added to the mixture at 25-30° C. and stirred for 1 hour. Cooled the mixture to −35 to −25° C. and stirred for 45 minutes. (3R,4S)-benzyl 3-(2-bromoacetyl)-4-ethylpyrrolidine-1-carboxylate (21.8 gms) and acetone (40 ml) was added to the mixture at −35 to −25° C. and stirred for 8 hours. Water and ethyl acetate were added to the mixture at −35 to −25° C. and stirred for 10 minutes. Raised the temperature of the mixture to −10 to −1° C. and stirred for 30 minutes. Layers were separated and aqueous layer was extracted with ethyl acetate. Combined the total organic layers and washed with aqueous sodium chloride solution. Distilled off the solvent completely from the organic layer to get the title compound. Yield: 26.5 gms.

### Example-2: Preparation of (3S,4R)-benzyl 3-ethyl-4-(3-tosyl-3H-imidazo[1,2-a]pyrrolo[2,3-e]pyrazin-8-yl)pyrrolidine-1-carboxylate formula-3a

Acetonitrile (500 ml) was added to (3R,4S)-benzyl 3-(2-((tert-butoxycarbonyl)(5-tosyl-5H-pyrrolo[2,3-b]pyrazin-2-yl)amino)acetyl)-4-ethylpyrrolidine-1-carboxylate (50 gms) at 25-30° C. and stirred for 10 minutes. Trifluoroacetic anhydride (35 ml) was added to the mixture at 25-30° C. and stirred for 15 minutes. Trifluoroacetic acid (10 ml) was added to the mixture at 25-30° C. and stirred for 15 minutes. Heated the mixture to 70-80° C. and stirred for 1 hour. Cooled the mixture to 25-30° C. and stirred for 15 minutes. Water and dichloromethane was added to the mixture at 25-30° C. and stirred for 15 minutes. Layers were separated and aqueous layer was extracted with ethyl acetate. Combined the total organic layers and washed with aqueous sodium chloride solution. Distilled off the solvent completely from the organic layer to get the title compound. Yield: 41.5 gms.

### Example-3: Preparation of 8-((3R,4S)-4-ethylpyrrolidin-3-yl)-3H-imidazo[1,2-a]pyrrolo[2,3-e]pyrazine formula-2

Isopropanol (250 ml) was added to (3S,4R)-benzyl 3-ethyl-4-(3-tosyl-3H-imidazo[1,2-a]pyrrolo[2,3-e]pyrazin-8-yl)pyrrolidine-1-carboxylate (50.0 gms) at 25-30° C. Water (500 ml) and barium hydroxide octa hydrate (290.10 gms) was added to the mixture at 25-30° C. and stirred for 10 minutes. Heated the mixture to 80-90° C. and stirred for 45-50 hours. Cooled the mixture to 25-30° C. and stirred for 15 minutes. Layers were separated and aqueous layer was extracted with a mixture of isopropanol and dichloromethane. Combined the total organic layers and washed with aqueous sodium chloride solution. Distilled off the solvent from the organic layer. Water was added to obtained compound at 25-30° C. Filtered the solid and washed with water and dried. To the obtained compound, dichloromethane was added at 25-30° C. Cooled the mixture to −15° C. to −20° C. and stirred for 1 hour. Filtered the solid, washed with dichloromethane and dried to get the title compound. Yield: 16.5 gms.

### Example-4: Preparation of 8-((3R,4S)-4-ethylpyrrolidin-3-yl)-3H-imidazo[1,2-a]pyrrolo[2,3-e]pyrazine

Isopropanol (1200 ml) was added to (3S,4R)-benzyl 3-ethyl-4-(3-tosyl-3H-imidazo[1,2-a]pyrrolo[2,3-e]pyrazin-8-yl)pyrrolidine-1-carboxylate (200.0 gms) obtained in example-2 at 25-30° C. Water (2400 ml) and barium hydroxide octa hydrate (1856.9 gms) added to the mixture at 25-30° C. and stirred for 10 minutes. Heated the mixture to 80-90° C. and stirred for 50 hours. Cooled the mixture to 25-30° C. and stirred for 15 minutes. Filtered the mixture. To the obtained compound, a mixture of isopropanol and dichloromethane was added. Filtered the unwanted solid and washed with mixture of isopropanol and dichloromethane. Combined filtrates and washed with aqueous sodium chloride solution. Filtered the mixture through hyflow bed and washed the bed with a mixture of isopropanol and dichloromethane. Combined the total organic layers and washed with aqueous sodium chloride solution. Distilled off the solvent from the organic layer. Water was added to the obtained compound and stirred for 2 hours at 25-30° C. Filtered the solid, washed with water and dried to get the title compound. Yield: 79.8 gms; Purity by HPLC is 98.83%.

The PXRD pattern of the obtained compound was depicted in FIG. 1.

### Example-5: Preparation of (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide

2,2,2-Trifluoroethylamine hydrochloride (3.18 gms) and carbonyldiimidazole (3.8 gms) were added to tetrahydrofuran (30.0 ml) at 25-30° C. and stirred for 10 minutes. Diisopropylethylamine (12.3 ml) was added to the mixture at 25-30° C. and stirred for 50 minutes. 8-((3R,4S)-4-ethylpyrrolidin-3-yl)-3H-imidazo[1,2-a]pyrrolo[2,3-e]pyrazine (3.0 gms) of formula-2 obtained in example-3 was added to the mixture at 25-30° C. and stirred for 20 hours. Water and ethyl acetate were added to the mixture at 25-30° C. and stirred for 20 minutes. Layers were separated and aqueous layer extracted with ethyl acetate. Combined the total organic layers and distilled off the solvent completely from the organic layer to get the title compound. Yield: 4.2 gms.

### Example-6: Preparation of (3S,4R)-benzyl 3-ethyl-4-(3H-imidazo[1,2-a]pyrrolo[2,3-e] pyrazin-8-yl)pyrrolidine-1-carboxylate of formula-3a(i)

Tetrahydrofuran (120 ml) was added to (3S,4R)-benzyl 3-ethyl-4-(3-tosyl-3H-imidazo [1,2-a]pyrrolo[2,3-e]pyrazin-8-yl)pyrrolidine-1-carboxylate (40 gms) of formula-3a at 25-30° C. Aqueous sodium hydroxide (200 ml) was added to the above mixture at 25-30° C. and stirred for 10 minutes. Heated the mixture to 60-70° C. and stirred for 3 hours. Cooled the mixture to 25-30° C. and stirred for 30 minutes. Layers were separated and aqueous layer extracted with ethyl acetate. Distilled off the organic layer. Ethyl acetate and water was added to the obtained compound at 25-30° C. and stirred for 10 minutes. Layers were separated. Organic layer was washed with aqueous sodium chloride solution. Distilled off the organic layer to get the title compound. Yield: 22.08 gms.

### Example-7: Preparation of (3S,4R)-benzyl 3-ethyl-4-(3H-imidazo[1,2-a]pyrrolo[2,3-e] pyrazin-8-yl)pyrrolidine-1-carboxylate of formula-3a(i)

Tetrahydrofuran (60 ml) was added to (3S,4R)-benzyl 3-ethyl-4-(3-tosyl-3H-imidazo [1,2-a]pyrrolo[2,3-e]pyrazin-8-yl)pyrrolidine-1-carboxylate (10 gms) of formula-3a at 25-30° C. Barium hydroxide octa hydrate (46.42 gms) and water (120 ml) were added above mixture at 25-30° C. and stirred for 10 minutes. Heated the mixture to 80-90° C. and stirred for 24 hours. Cooled the mixture to 25-30° C. and stirred for 30 minutes. Dichloromethane (30 ml) was added to the mixture at 25-30° C. and stirred for 10 minutes. Filtered the mixture and washed with a mixture of isopropanol and dichloromethane. Layers were separated from filtrate and aqueous layer was extracted with a mixture of isopropanol and dichloromethane. Distilled off the solvent completely from the organic layer to get the title compound. Yield: 5.75 gms.

### Example-8: Preparation of 8-((3R,4S)-4-ethylpyrrolidin-3-yl)-3H-imidazo[1,2-a]pyrrolo [2,3-e]pyrazine formula-2

Isopropanol (60.0 ml) was added to (3S,4R)-benzyl 3-ethyl-4-(3H-imidazo[1,2-a] pyrrolo[2,3-e]pyrazin-8-yl)pyrrolidine-1-carboxylate (10.0 gms) of formula-3a(i) obtained in Example-6 at 25-30° C. Water (120 ml) and barium hydroxide octa hydrate (64.8 gms) were added to the mixture at 25-30° C. and stirred for 10 minutes. Heated the mixture to 80-90° C. and stirred for 24 hours. Cooled the mixture to 25-30° C. and stirred for 15 minutes. Filtered the mixture. To the obtained compound, a mixture of isopropanol and dichloromethane was added. Filtered the solid and washed with a mixture of isopropanol and dichloromethane. Combined filtrates and washed with aqueous sodium chloride solution. Filtered the mixture through hyflow bed and washed the bed with a mixture of isopropanol and dichloromethane. Combined the total organic layers and washed with aqueous sodium chloride solution. Distilled off the organic layer. Water was added to the obtained compound and stirred for 3 hours. Filtered the solid, washed with water and dried to get the title compound.

Yield: 3.8 gms.

### Example-9: Preparation of Dibenzoyl-L-Tartaric Acid Salt of Upadacitinib

Isopropanol (6.0 ml) was added to Upadacitinib (5.0 gms) of compound of formula-1 at 25-30° C. and stirred for 10 minutes. Water (3.6 ml) was added to the mixture at 25-30° C. and stirred for 10 minutes. Isopropyl acetate (95.0 ml) was added to the mixture at 25-30° C. and stirred for 10 minutes. Dibenzoyl-L-tartaric acid (5.4 gms) was added to the mixture at 25-30° C. and stirred for 10 minutes. Heated the mixture to 45-55° C. and stirred for 1 hour. Cooled the mixture to 25-30° C. Filtered the solid and washed with isopropyl acetate to get the titled compound. Yield: 8.4 gms.

The PXRD of the obtained compound was depicted in FIG. 2.

### Example-10: Preparation of Upadacitinib

Ethyl acetate (35.0 ml) and water (35.0 ml) were added to Dibenzoyl-L-tartaric acid salt of Upadacitinib (8.4 gms) at 25-30° C. and stirred for 10 minutes. Cooled the mixture to 5-15° C. and added aqueous sodium carbonate solution at same temperature. Layers were separated and aqueous layer was extracted with ethyl acetate. Combined the total organic layers and washed with aqueous sodium chloride solution. Distilled off the solvent completely from the organic layer to get the title compound.

Yield: 4.0 gms; Purity by HPLC of 99.80%.

### Example-11: Preparation of 4-Nitrobenzoic Acid Salt of Upadacitinib

4-Nitrobenzoic acid (1.3 gms) and acetonitrile (10.0 ml) were added to Upadacitinib (2.0 gms) of compound of formula-1 at 25-30° C. and stirred for 2 hours. Filtered the solid and washed with acetonitrile to get the titled compound. Yield: 3.3 gms.

The PXRD pattern of the obtained compound is depicted in FIG. 3.

### Example-12: Preparation of Upadacitinib

Ethyl acetate (14.0 ml) and water (14.0 ml) were added to 4-nitrobenzoic acid salt of Upadacitinib (3.3 gms) at 25-30° C. and stirred for 10 minutes. Cooled the mixture to 5-15° C. and added sodium carbonate at same temperature. Layers were separated and aqueous layer was extracted with ethyl acetate. Combined the total organic layers and washed with aqueous sodium chloride solution. Distilled off the solvent completely from the organic layer to get the title compound. Yield: 1.0 gms; Purity of HPLC of 99.62%.

### Example-13: Preparation of Dibenzoyl-L-Tartaric Acid Salt of Upadacitinib

2,2,2-Trifluoroethylamine hydrochloride (55.7 gms) was added to tetrahydrofuran (500.0 ml) at 25-30° C. and stirred for 10 minutes. Cooled the mixture to 0-5° C. and added carbonyldiimidazole (60.33 gms) lot wise at same temperature and stirred for 10 minutes. Cooled the mixture to −5° C. to 0° C. Diisopropylethylamine (136.8 ml) was added to the mixture at −6° C. to 0° C. Heated the mixture to 25-30° C. and stirred for 3 hours. 8-((3R,4S)-4-ethylpyrrolidin-3-yl)-3H-imidazo[1,2-a]pyrrolo[2,3-e]pyrazine (50.0 gms) of formula-2 was added to the mixture at 25-30° C. and stirred for 4 hours. Water (250 ml) and ethyl acetate (300 ml) were added to the mixture at 25-30° C. and stirred for 20 minutes. Layers were separated and aqueous layer was extracted with ethyl acetate. Combined the total organic layers and extracted with aqueous ortho phosphoric acid solution. Cooled the mixture to 5-15° C. Aqueous sodium carbonate solution was added to the mixture at 5-15° C. Ethyl acetate was added to the mixture. Raised the temperature to 25-30° C. Layers we separated and extracted the aqueous layer with ethyl acetate. Combined the organic layers and distilled off the solvent completely. To the obtained compound, acetonitrile (650.0 ml) was added to at 25-30° C. and stirred for 10 minutes. Water (31.0 ml) was added to the mixture at 25-30° C. and stirred for 10 minutes. A solution of acetonitrile (100.0 ml) and dibenzoyl-L-tartaric acid (63.17 g) was added to the mixture at 25-30° C. and stirred for 10 minutes. Raised the temperature of the mixture to 55-65° C. and stirred for 30 minutes. Cooled the mixture to 25-30° C. and stirred for 3 hours. Filtered the solid and washed with acetonitrile. Water was added to the obtained solid. Cooled to 5-15° C. and stirred for 10 minutes. Aqueous sodium carbonate solution was added to the mixture at 5-15° C. and stirred for 10 minutes. Raised the temperature of the mixture to 25-30° C. Layer are separated and extracted the aqueous layer with ethyl acetate. Combined the organic layers and distilled of the solvent completely under reduced pressure. To the obtained compound, acetonitrile (650.0 ml) was added to at 25-30° C. and stirred for 10 minutes. Water (31.0 ml) was added to the mixture at 25-30° C. and stirred for 10 minutes. A solution of acetonitrile (100.0 ml) and dibenzoyl-L-tartaric acid (63.17 gms) was added to the mixture at 25-30° C. and stirred for 10 minutes. Heated the mixture to 55-65° C. and stirred for 30 minutes. Cooled the mixture to 25-30° C. and stirred for 3 hours. Filtered the solid, washed with acetonitrile and dried to get the titled compound. Yield: 94.0 gms.

The PXRD pattern of the obtained compound is depicted in FIG. 4.

### Example-14: Preparation of (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide formula-1

Water (600.0 ml) was added to dibenzoyl-L-tartaric acid salt of Upadacitinib (150.0 gms) at 25-30° C. and stirred for 10 minutes. Cooled the mixture to 5-15° C. Ethyl acetate (900.0 ml) was added at 5-15° C. Layers were separated and aqueous layer was extracted with ethyl acetate. Combined the total organic layers washed with aqueous sodium carbonate solution. Sodium sulfate was added to the organic layers. Filtered the mixture and washed with ethyl acetate. Distilled off the solvent completely from the organic layer. Ethanol (30 ml) was added to the obtained compound at 25-30° C. and stirred for 10 minutes. Filtered the mixture through hyflow bed and washed with ethanol. The obtained filtrate was added to pre-cooled water (600 ml) at 0-5° C. and stirred for 10 minutes. Filtered the solid, washed with pre-cooled water and dried to get the titled compound. Yield: 61.0 gms; Purity by HPLC: 99.84%; Des tosyl impurity: 0.05%; Chiral Purity by HPLC: 99.99%; Enantiomer impurity: Not detected. The PXRD pattern of the obtained compound is depicted in FIG. 6.

Particle size distribution (PSD): D(10): 7.86 μm; D(50): 58.19 μm; D(90): 190.89 μm.

### Example-15: Preparation of Crystalline Form-M of Upadacitinib Tartrate

Ethyl acetate (1.0 ml) was added to Upadacitinib (100.0 mg) of compound of formula-1 at 25-30° C. and stirred for 10 minutes to get mixture-a. Mixture of methyl tert-butyl ether and methyl isobutyl ketone (1.0 ml) were added to tartaric acid (39.5 mg) at 25-30° C. and cooled the temperature to 0-5° C. to get mixture-B. Adding mixture-A in mixture-B at 0-5° C. and stirred for 4 hours. Filtered the solid under vacuum to get the title compound.

Yield: 80.0 mg; The PXRD pattern of the obtained compound is depicted in FIG. 5.

### Example-16: Preparation of Crystalline Form-M of Upadacitinib Tartrate

Ethyl acetate (0.5 ml) was added to Upadacitinib (50.0 mg) of compound of formula-1 at 28° C. and stirred for 10 minutes to mixture-A. Mixture of methyl tert-butyl ether and methyl isobutyl ketone (1.0 ml) were added to tartaric acid (19.5 mg) at 28° C. and cooled the temperature to 0-5° C. to get mixture-B. Adding mixture-A in mixture-B at 0-5° C. and stirred for 4 hours. Filtered the solid under vacuum to get the title compound. Yield: 45.0 mg.

The PXRD pattern of the obtained compound was depicted in FIG. 5.

### Example-17: Preparation of (3R, 4S)-benzyl 3-(2-((tert-butoxycarbonyl)(5-tosyl-5H-pyrrolo[2,3-b] pyrazin-2-yl) amino) acetyl)-4-ethylpyrrolidine-1-carboxylate formula-4

Acetone (1300.0 ml) was added to tert-butyl (5-tosyl-5H-pyrrolo[2,3-b] pyrazin-2-yl) carbamate (100 gms) of formula-5 at 25-30° C. and stirred for 10 min. Cesium carbonate (251.6 gms) and tetrabutylammonium bromide (41.49 gms) were added to the mixture at 25-30° C. and stirred for 1 hour. Molecular sieves (25.0) were added to the mixture at 25-30° C. and stirred for 1 hour. Cooled the mixture to −35 to −25° C. and stirred for 45 minutes. (3R, 4S)-benzyl 3-(2-bromoacetyl)-4-ethylpyrrolidine-1-carboxylate (109.4 gms) and acetone (200 ml) was added to the mixture at −35 to −25° C. and stirred for 8 hours. Water (200 ml) and ethyl acetate (1000 ml) were added to the mixture at −35 to −25° C. and stirred for 10 minutes. Raised the temperature of the mixture to −10 to 0° C. and stirred for 30 minutes. Layers were separated and aqueous layer was extracted with ethyl acetate. Combined the total organic layers and washed with aqueous sodium chloride solution. Distilled off the solvent completely from the organic layer to get the title compound. Yield: 149.0 gms.

### Example-18: Preparation of (3S,4R)-benzyl 3-ethyl-4-(3-tosyl-3H-imidazo[1,2-a]pyrrolo [2,3-e]pyrazin-8-yl)pyrrolidine-1-carboxylate formula-3a

Acetonitrile (1000 ml) was added to (3R, 4S)-benzyl 3-(2-((tert-butoxycarbonyl) (5-tosyl-5H-pyrrolo[2,3-b] pyrazin-2-yl) amino) acetyl)-4-ethylpyrrolidine-1-carboxylate (100 gms) at 25-30° C. and stirred for 10 minutes. Trifluoroacetic anhydride (80 ml) was added to the mixture at 25-30° C. and stirred for 15 minutes. Trifluoroacetic acid (20 ml) was added to the mixture at 25-30° C. and stirred for 15 minutes. Heated the mixture to 75-85° C. and stirred for 1 hour. Cooled the mixture to 25-30° C. and stirred for 15 minutes. Water (500 ml) and dichloromethane (500 ml) added to the mixture at 25-30° C. and stirred for 15 minutes. Layers were separated and aqueous layer was extracted with ethyl acetate. Combined the total organic layers and washed with aqueous sodium chloride solution. Distilled off the solvent completely from the organic layer to get the title compound. Yield: 64.0 gms.

We claim:

1. A process for preparation of compound of formula-2, comprising treating the compound of general formula-3 with a base. 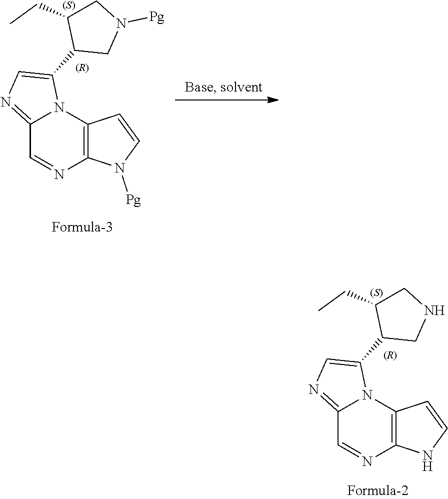
wherein, Pg refers to a protecting group.

2. The process as claimed in claim 1, wherein the base is selected from sodium hydroxide, potassium hydroxide, cesium carbonate, sodium carbonate, potassium carbonate, lithium carbonate, rubidium carbonate, sodium hydroxide, potassium hydroxide, lithium hydroxide, barium hydroxide, barium hydroxide octa hydrate, caesium hydroxide, strontium hydroxide, calcium hydroxide, pyridine thereof.

3. The process as claimed in claim 1, wherein the solvent is selected from methanol, ethanol, n-propanol, isopropanol, n-butanol, isobutanol, t-butanol, n-pentanol, isopentanol, 2-nitroethanol, water and/or mixtures thereof.

4. The process as claimed in claim 1, wherein the protecting group selected from carbobenzyloxy (Cbz), benzoyl (Bz), benzyl (Bn), tosyl (Ts), p-methoxybenzyl carbonyl, tert-butyloxycarbonyl (BOC), acetyl (Ac), carbamate, p-methoxybenzyl, 3,4-dimethoxybenzyl, p-methoxyphenyl (PMP), trichloroethylchloroformate, nosyl thereof.

5. A process for preparation of 8-((3R,4S)-4-ethylpyrrolidin-3-yl)-3H-imidazo[1,2-a] pyrrolo[2,3-e]pyrazine compound of formula-2, comprising treating (3S,4R)-benzyl 3-ethyl-4-(3-tosyl-3H-imidazo[1,2-a]pyrrolo[2,3-e]pyrazin-8-yl)pyrrolidine-1-carboxylate of formula-3a with barium hydroxide octa hydrate in a mixture of isopropanol and water to provide 8-((3R,4S)-4-ethylpyrrolidin-3-yl)-3H-imidazo[1,2-a]pyrrolo[2,3-e]pyrazine compound of formula-2.

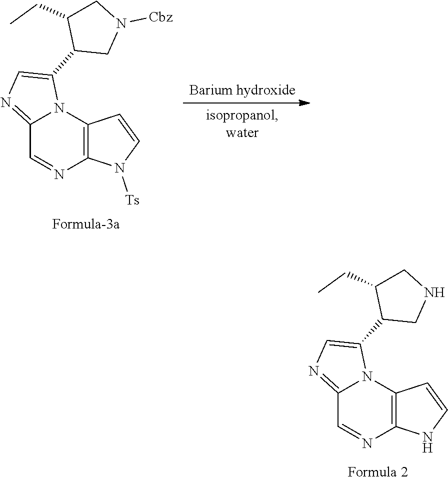

6. The process as claimed in claims 1 and 5 wherein, barium hydroxide octa hydrate used is 2 moles to 20 moles with respect to 1 mole of compound of general formula-3 and formula-3.

7. The process as claimed in claims 1 and 5 wherein, barium hydroxide octa hydrate used preferably is 8 moles to 16 moles with respect to 1 mole of compound of general formula-3 and formula-3.

8. A process for preparation of compound of formula-2, comprising reacting the compound of general formula-3(i) with base to provide compound of formula-2.

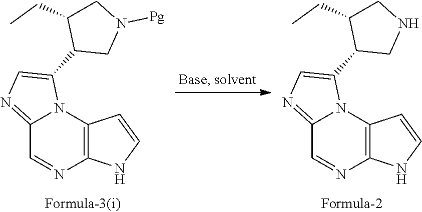

9. The process as claimed in claim 8, wherein the base is selected from sodium hydroxide, potassium hydroxide, cesium carbonate, sodium carbonate, potassium carbonate, lithium carbonate, rubidium carbonate, sodium hydroxide, potassium hydroxide, lithium hydroxide, barium hydroxide, barium hydroxide octa hydrate, caesium hydroxide, strontium hydroxide, calcium hydroxide, pyridine thereof.

10. The process as claimed in claim 8, wherein the solvent is selected from methanol, ethanol, n-propanol, isopropanol, n-butanol, isobutanol, t-butanol, n-pentanol, isopentanol, 2-nitroethanol, water and/or mixtures thereof.

11. The process as claimed in claim 8, wherein the protecting group selected from carbobenzyloxy (Cbz), benzoyl (Bz), benzyl (Bn), tosyl (Ts), p-methoxybenzyl carbonyl (Moz or MeOZ), tert-butyloxycarbonyl (BOC), acetyl (Ac), carbamate, p-methoxybenzyl, 3,4-dimethoxybenzyl, p-methoxyphenyl (PMP), trichloroethyl chloroformate, nosyl thereof.

12. A process for the preparation of compound of formula-2, comprising, reacting (3S,4R)-benzyl 3-ethyl-4-(3H-imidazo[1,2-a]pyrrolo[2,3-e]pyrazin-8-yl)pyrrolidine-1-carboxylate of formula-3a(i) with barium hydroxide octa hydrate in a mixture of isopropanol and water to provide 8-((3R,4S)-4-ethylpyrrolidin-3-yl)-3H-imidazo[1,2-a]pyrrolo[2,3-e]pyrazine compound of formula-2.

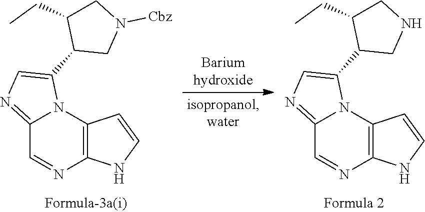

13. The process as claimed in claims 8 and 12 wherein, barium hydroxide octa hydrate used is from 1 moles to 10 moles with respect to 1 mole of compound of general formula-3(i) and formula-3a(i).

14. Crystalline form of 8-((3R,4S)-4-ethylpyrrolidin-3-yl)-3H-imidazo[1,2-a]pyrrolo[2,3-e] pyrazine.

15. Crystalline form of 8-((3R,4S)-4-ethylpyrrolidin-3-yl)-3H-imidazo[1,2-a]pyrrolo[2,3-e] pyrazine compound of formula-2, is characterized by its powder X-Ray diffractogram as illustrated in FIG. 1.

16. 8-((3R,4S)-4-ethylpyrrolidin-3-yl)-3H-imidazo[1,2-a]pyrrolo[2,3-e]pyrazine having purity of at least about 98.80% as measured by HPLC.

17. Dibenzoyl-L-tartaric acid salt of Upadacitinib.

18. Dibenzoyl-L-tartaric acid salt of Upadacitinib of claim 17, wherein the salt is crystalline.

19. Crystalline Form-I of Upadacitinib Dibenzoyl-L-tartaric acid salt of claim 18, is characterized by its powder X-Ray diffractogram as illustrated in FIG. 2.

20. Crystalline Form-II of Upadacitinib Dibenzoyl-L-tartaric acid salt of claim 18, is characterized by its powder X-Ray diffractogram as illustrated in FIG. 4.

21. A process for the preparation of crystalline Form-I of dibenzoyl-L-tartaric acid salt of Upadacitinib, comprising:
a) treating the compound of formula-1 with dibenzoyl-L-tartaric acid in isopropanol, water and isopropyl acetate,
b) heating the mixture obtained in step-a) to a temperature ranging from 25° C. to 60° C.,
c) isolating crystalline Form-I of dibenzoyl-L-tartaric acid salt of Upadacitinib.

22. A process for the preparation of crystalline Form-II of dibenzoyl-L-tartaric acid salt of Upadacitinib, comprising:
a) treating the compound of formula-1 with dibenzoyl-L-tartaric acid in acetonitrile and water,
b) heating the mixture obtained in step-a) to a temperature ranging from 25° C. to 65° C.,
c) isolating crystalline Form-II of dibenzoyl-L-tartaric acid salt of Upadacitinib.

23. 4-Nitrobenzoic acid salt of Upadacitinib.

24. 4-Nitrobenzoic acid salt of Upadacitinib of claim 23, wherein the salt is crystalline.

25. Crystalline Form-I of Upadacitinib 4-nitrobenzoic acid salt of claim 24, is characterized by its powder X-Ray diffractogram as illustrated in FIG. 3.

26. A process for the preparation of crystalline form of 4-nitrobenzoic acid salt of Upadacitinib, comprising:
a) treating the compound of formula-1 with 4-nitrobenzoic acid in acetonitrile to provide 4-nitrobenzoic acid salt of Upadacitinib,
b) isolating crystalline form of 4-nitrobenzoic acid salt of Upadacitinib.

27. Crystalline form-M of Upadacitinib tartrate is characterized by its powder X-Ray diffractogram as illustrated in FIG. 5.

28. A process for the preparation of crystalline Form-M of Upadacitinib tartrate, which comprises:
a) providing a solution of Upadacitinib of formula-1,
b) providing a solution of tartaric acid,
c) adding the solution obtained in step-a) to the solution obtained in step-b) to provide crystalline Form-M of Upadacitinib tartrate.

29. The process as claimed in claim 28, wherein the solvent used in step-a) is ester solvent which is selected from methyl acetate, ethyl acetate, isopropyl acetate, n-butyl acetate, isobutyl acetate or mixture thereof.

30. The process as claimed in claim 28, wherein providing a solution of Upadacitinib of step-a) comprises dissolving Upadacitinib in ethyl acetate. Optionally filtering the mixture to make it particle free.

31. The process as claimed in claim 28, wherein the solvent used in step-b) is ether solvent is selected from dimethyl ether, diisopropyl ether, diethyl ether, methyl tert-butyl ether, 1,2-dimethoxyethane, tetrahydrofuran, ketone solvent selected from acetone, methyl ethyl ketone, methyl isobutyl ketone or mixture thereof.

32. The process as claimed in claim 28, wherein in step-c), adding solution obtained in step-a) in solution obtained in step-b) at a temperature ranging from 0° C. to 15° C.

33. A process for the preparation of pure Upadacitinib of formula-1, comprises:
a) reacting compound of general formula-4 with trifluoro acetic acid and trifluoro acetic anhydride in acetonitrile in the absence of base to provide compound of general formula-3,
b) treating the compound of general formula-3 with a suitable base to provide compound of formula-2,
c) reacting the compound of formula-2 with 2,2,2-trifluoroethanamine hydrochloride in the presence of coupling agent and a base and followed by treating the obtained compound with an acid to provide acid addition salt of Upadacitinib,
d) treating the acid addition salt of Upadacitnib with a base to provide pure Upadacitinib.

34. The process as claimed in claim 33, wherein
the suitable base used in step-b) and step-d) is selected from sodium hydroxide, potassium hydroxide, cesium carbonate, sodium carbonate, potassium carbonate, lithium carbonate, sodium hydroxide, potassium hydroxide, lithium hydroxide, barium hydroxide, barium hydroxide octa hydrate, caesium hydroxide, calcium hydroxide;
the suitable coupling agent used in step-c) is selected from EDCI·HCl, HATU, DCC and CDI with or without HOBt or HOEt or in a combination thereof, the suitable base selected from triethyl amine, diisopropylethyl amine, dimethylaminopyridine;
the suitable acid used in step-c) is selected form dibenzoyl-L-tartaric acid and 4-nitrobenzoic acid;
the suitable solvent used in step-a) to step-d) is selected from ketone solvents, ester solvents, chloro solvents, alcohol solvents, ether solvents, nitrile solvents, polar aprotic solvents, hydrocarbon solvents and water or mixture thereof.

35. Upadacitinib obtained according to the any of preceding claims is having purity of at least about 95%; preferably of at least about 97%; more preferably of at least about 98%; most preferably of at least about 99.9% as measured by HPLC.

36. Upadacitinib obtained according to the any of preceding claims is having chiral purity of at least about 95%; preferably of at least about 97%; more preferably of at least about 98%; most preferably of at least about 99.9% as measured by HPLC.

37. Amorphous form of Upadacitinib obtained according to the preceding claims is having purity of greater than about 99.80% as measured by HPLC.

38. Upadacitinib obtained according to the preceding claims is having particle size distribution D₉₀<250 μm.

39. Upadacitinib obtained according to the preceding claims is useful for the preparation of pharmaceutical composition.

40. A pharmaceutical composition comprising Upadacitinib according to the preceding claims and one or more pharmaceutically acceptable excipients.
